(19) United States
(12) Patent Application Publication
(10) Pub. No.: US 2007/0155632 A1
(43) Pub. Date: Jul. 5, 2007

(54) Lubricating oil composition for continuously variable transmission

(75) Inventors: Hitoshi Hata, Chiba (JP); Hidetoshi Koga, Chiba (JP); Tomomi Miyaji, Chiba (JP); Toshiyuki Tsubouchi, Chiba (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Idemitsu Kosan Co., Ltd., Tokyo (JP)

(21) Appl. No.: 10/594,764

(22) PCT Filed: Mar. 29, 2005
(86) PCT No.: PCT/JP05/05886

§ 371 Date: Sep. 29, 2006

(30) Foreign Application Priority Data
Mar. 29, 2004 (JP) 2004-097176

(51) Int. Cl. C07D 251/54

(52) U.S. Cl. 508/258

(57) ABSTRACT

There is provided a lubricating oil composition for continuously variable transmission which is capable of enhancing a power transmission capacity of CVTs of various type including a metallic belt type, a chain type and a traction drive type, and keeping the enhanced power transmission capacity. The lubricating oil composition for continuously variable transmission according to the present invention includes (A) a base oil comprising a hydrocarbon compound having a cohesive energy density at 40° C. of 0.180 GPa or more, and having a kinematic viscosity at 40° C. of 5 to 150 mm²/s, and (B) a phosphoric ester containing a alkyl group having a thioether bond, and/or (C) at least one compound selected from the group consisting of phosphoric esters and amine salts thereof, and an overbased calcium sulfonate.

### TECHNICAL FIELD

The present invention relates to lubricating oil compositions for continuously variable transmission, and more particularly to lubricating oil compositions for continuously variable transmission which are generally applicable to metallic belt type continuously variable transmissions, chain type continuously variable transmissions and traction drive type continuously variable transmissions.

### BACKGROUND ART

There are known continuously variable transmissions (hereinafter occasionally referred to merely as “CVT”) of various types, for example, a metallic belt type, a chain type and a traction drive type. Although any type of CVTs is required to have a high power transmission capacity, the power transmission performance of these CVTs varies depending upon properties of a lubricating oil used therein, more specifically, a metal-to-metal friction coefficient or a traction coefficient. The larger the both coefficients, the higher the power transmission capacity becomes.

The lubricating condition of the power-transmitting surface (a contact surface between metals) of the CVTs is considered to be a mixed lubricating condition including both an elastohydrodynamic lubrication (EHL) and a boundary/extreme pressure lubrication. In the metallic belt type CVT and the chain type CVT, the power-transmitting surface is mainly kept under the boundary/extreme pressure lubrication condition and, therefore, it is considered that the power-transmitting capacity thereof mainly depends upon the metal-to-metal friction coefficient. For this reason, as a base oil of the lubricating oil for CVTs of these types, there have been conventionally used hydrocarbon compounds such as paraffinic mineral oils and poly-α-olefins (PAO) to which additives are added to suitably control the metal-to-metal friction coefficient. On the other hand, in conventional traction drive type CVT, since the power transmission is performed through an EHL oil film, the power transmission capacity mainly depends upon the traction coefficient. Therefore, as a base oil of the lubricating oil for the traction drive type CVT, there have been conventionally used synthetic naphthenic compounds having a high traction coefficient. Thus, in CVTs of various type, special lubricating oils have been individually used (for example, refer to Japanese Patent Application Laid-open Nos. 09-100487 and 2001-288488).

However, in the metallic belt type CVT or the chain type CVT, the traction coefficient thereof is inherently small, and further the power transmission surface tends to undergo the change in shape of the friction surface when worn out by friction, thereby causing significant change in lubrication condition of the contact surface, etc. As a result, the metal-to-metal friction coefficient tends to be lowered, resulting in failure to accomplish desired power transmission and exhibit a good CVT function.

In addition, in the traction drive type CVT, the power transmission is partially performed in the boundary/extreme pressure lubrication manner under high-temperature and high-load conditions, so that the metal-to-metal friction coefficient at the contact surface tends to become unsatisfactory. As a result, the friction coefficient on the power-transmitting surface tends to be lowered, resulting in failure to attain desired power transmission, as well as less functional durability of the CVT.

For these reasons, it has been demanded to enhance a power transmission capacity of the respective types of CVTs, and improve a durability of CVT units by maintaining the enhanced power transmission capacity.

### DISCLOSURE OF THE INVENTION

### Problem to be Solved by the Invention

The present invention has been made in view of the above problems. An object of the present invention is to provide a lubricating oil composition for continuously variable transmission which is capable of enhancing a power transmission capacity of CVTs of various types such as a metallic belt type, a chain type and a traction drive type, and maintaining the enhanced power transmission capacity.

### MEANS FOR SOLVING PROBLEM

The present inventors have found that when controlling a cohesive energy density of a base oil to a predetermined value or more, a metal-to-metal friction coefficient imparted by additives added thereto can be enhanced, and the enhanced friction coefficient can be maintained, and at the same time, such a base oil exhibits a high traction coefficient. The present invention has been accomplished on the basis of the finding.

Thus, the present invention provides:

[1] A lubricating oil composition for continuously variable transmission comprising:

(A) a base oil comprising a hydrocarbon compound having a cohesive energy density at 40° C. of 0.180 GPa or more, said base oil having a kinematic viscosity at 40° C. of 5 to 150 mm²/s, and

(B) a phosphoric ester containing a hydrocarbon group having a thioether bond, and/or (C) at least one compound selected from the group consisting of phosphoric esters and amine salts thereof, and an overbased calcium sulfonate.

[2] A lubricating oil composition for continuously variable transmission as described in the above aspect [1], wherein said cohesive energy density (CED) at 40° C. is calculated from the following formula (1):
CED(GPa)=0.0204(d/MW)·T·ln(2.51η·MW) (1)
wherein d is a density (g/cm³) at 40° C.; MW is a molecular weight (g/mol); T is an absolute temperature (K); and η is a kinematic viscosity (mm²/s) at 40° C.

[3] A lubricating oil composition for continuously variable transmission as described in the above aspect [1] or [2], wherein said base oil (A) contains at least one compound selected from the group consisting of (i) dimerized norbornanes, (ii) hydrogenated dimers, trimers and tetramers of norbornanes and/or norbornenes, (iii) alkane derivatives having 2 or more cyclohexane rings; and (iv) alkane derivatives having one or more decalin rings and one or more cyclohexane rings.

[4] A lubricating oil composition for continuously variable transmission as described in any one of the above aspects [1] to [3], wherein said phosphoric ester (B) containing a hydrocarbon group having a thioether bond is an acid phosphoric ester or phosphorous ester represented by the general formula (II): 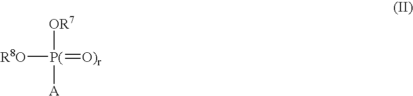
wherein r is 0 or 1; when r is 0, A is a hydroxyl group, and when r is 1, A is a hydrogen atom or a hydroxyl group; and R⁷ and R⁸ are respectively a hydrogen atom or a hydrocarbon group having 1 to 18 carbon atoms which may have a thioether bond with the proviso that at least one of R⁷ and R⁸ is the hydrocarbon group having a thioether bond.

[5] A lubricating oil composition for continuously variable transmission as described in any one of the above aspects [1] to [4], wherein said phosphoric esters and amine salts thereof as the component (C) are phosphoric esters containing an alkyl group having 3 to 12 carbon atoms or an aryl group having 6 to 12 carbon atoms, and amine salts thereof.

[6] A lubricating oil composition for continuously variable transmission as described in any one of the above aspects [1] to [5], wherein said overbased calcium sulfonate as the component (C) has a base value of 50 to 700 mg KOH/g.

[7] A lubricating oil composition for continuously variable transmission as described in any one of the above aspects [1] to [6], further comprising (D) a sulfur containing anti-wear agent.

[8] A lubricating oil composition for continuously variable transmission as described in any one of the above aspects [1] to [7], wherein said continuously variable transmission is of a metallic belt type.

[9] A lubricating oil composition for continuously variable transmission as described in any one of the above aspects [1] to [7], wherein said continuously variable transmission is of a chain type.

[10] A lubricating oil composition for continuously variable transmission as described in any one of the above aspects [1] to [7], wherein said continuously variable transmission is of a traction drive type.

### EFFECT OF THE INVENTION

The lubricating oil composition for continuously variable transmission according to the present invention is capable of enhancing a friction coefficient at a contact surface of a metallic belt type CVT, a chain type CVT, a traction drive type CVT, etc., exhibiting an excellent wear resistance and, therefore, imparting a large power transmission capacity and a high durability to these CVTs. In addition, the lubricating oil composition for continuously variable transmission according to the present invention is a general-purpose lubricating oil for continuously variable transmission which is applicable to various types of CVTs.

### PREFERRED EMBODIMENTS FOR CARRYING OUT THE INVENTION

The base oil used as the component (A) in the present invention is made of a hydrocarbon compound having a cohesive energy density (hereinafter occasionally referred to merely as “CED”) at 40° C. of 0.180 GPa or more and preferably 0.200 GPa or more. When the CED at 40° C. is 0.180 GPa or more, the base oil itself can exhibit a high traction coefficient, and further a composition obtained by blending the base oil with the component (B), etc., can exhibit a high metal-to-metal friction coefficient and at the same time, the effect of enhancing a wear resistance, so that the continuously variable transmission can be enhanced in power transmission capacity as well as persistency of the enhanced power transmission capacity. The CED at 40° C. may be determined, for example, according to the following formula (1):
CED(GPa)=0.0204(d/MW)·T·ln(2.51ηMW) (1)

wherein d is a density (g/cm³) at 40° C.; MW is a molecular weight (g/mol); T is an absolute temperature (K) i.e. T=303K; and η is a kinematic viscosity (mm²/s) at 40° C. Therefore, the CED at 40° C. can be calculated by substituting the respective measured values for corresponding terms in the above formula (1).

Also, the base oil used as the component (A) in the present invention has a kinematic viscosity at 40° C. of 5 to 150 mm²/s and preferably 8 to 50 mm²/s. When the kinematic viscosity at 40° C. of the base oil is 5 mm²/s or more, the resultant composition can prevent wear of various mechanical elements of CVT or CVT units such as gears and bearings. When the kinematic viscosity at 40° C. of the base oil is 150 mm²/s or less, the resultant composition is capable of preventing deterioration in low-temperature startability.

The base oil used as the component (A) in the present invention is not particularly limited, and various base oils may be used as long as they satisfy the above requirements. Examples of the suitable base oil include synthetic alicyclic (cyclic saturated) hydrocarbon compounds. The synthetic alicyclic hydrocarbon compounds used herein means those compounds including naphthenic compounds and condensed ring compounds. Examples of the synthetic alicyclic hydrocarbon compounds includes the following compounds (i) to (iv):

(i) dimerized norbornanes;

(ii) hydrogenated dimers, trimers and tetramers of norbornanes and/or norbornenes;

(iii) alkane derivatives having 2 or more cyclohexane rings; and

(iv) alkane derivatives having one or more decalin rings and one or more cyclohexane rings.

Examples of the above dimerized norbornanes (i) include those compounds represented by the general formula (I): 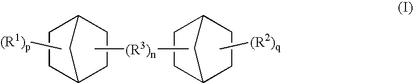
wherein R¹ and R² are respectively a hydrogen atom or an alkyl group having 1 to 3 carbon atoms; R³ is a methylene, ethylene or trimethylene group which may have a methyl substituent group bonded to a side chain thereof; n is 0 or 1; p and q are respectively an integer of 1 to 3 with the proviso that a sum of p and q (p+q) is an integer of 4 or less. These norbornanes are generally classified into two types according to the number n. That is, when n is 0, the general formula (I) is expressed by the following formula (I′): 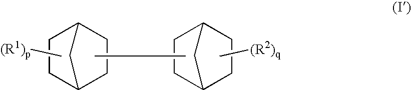
When n is 1, the general formula (I) is expressed by the following formula (I″):

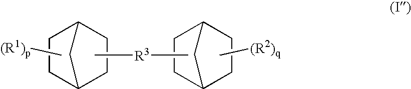

Meanwhile, in the general formulae (I′) and (I″), R¹ and R² are respectively a hydrogen atom or an alkyl group having 1 to 3 carbon atoms (such as methyl, ethyl, n-propyl and i-propyl); R³ is methylene, ethylene, trimethylene or these groups having a methyl group bonded to a side chain thereof (e.g., an ethylidene group and a methyl ethylene group); and p and q are respectively an integer of 1 to 3 with the proviso that a sum of p and q (p+q) is an integer of 4 or less.

These compounds represented by the general formulae (I′) and (I″) may be produced by various methods, and the production method therefore is not particularly limited. The compounds represented by the general formulae (I′) and (I″) may be usually produced by dimerizing norbornanes and/or norbornenes and then hydrogenating the resultant dimers.

The hydrogenated dimers, trimers and tetramers (ii) of norbornanes and/or norbornenes are hydrogenated products of dimers, trimers and tetramers (except for homopolymers of cyclic monoterpenoids) of either one or both of the norbornanes and norbornenes. As the norbornanes and norbornenes used herein as a raw material of the dimers, trimers or tetramers, there may be used various norbornanes and norbornenes without any particular limitations. Examples of the preferred norbornanes include those compounds represented by the following general formula: 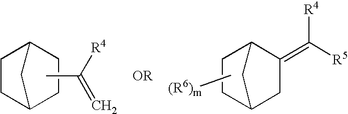
wherein R⁴, R⁵ and R⁶ are respectively a hydrogen atom or an alkyl group having 1 to 3 carbon atoms and preferably a hydrogen atom or methyl; and m is 1 or 2. Specific examples of the norbornanes include alkenyl norbornanes such as vinyl norbornane and isopropenyl norbornane; and alkylidene norbornanes such as methylene norbornane, ethylidene norbornane, isopropylidene norbornane, 3-methyl-2-methylene norbornane and 3,3-dimethyl-2-methylene norbornane. Examples of the preferred norbornenes includes those compounds represented by the following general formula: 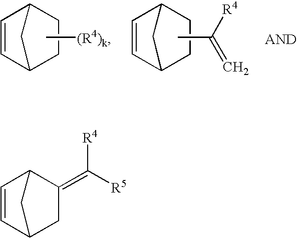
wherein R⁴ and R⁵ are the same as defined above; and k is 1 or 2. Specific examples of the norbornenes include, in addition to norbornene, alkyl norbornenes such as methyl norbornene, ethyl norbornene, isopropyl norbornene and dimethyl norbornene; alkenyl norbornenes such as vinyl norbornene and isopropenyl norbornene; and alkylidene norbornenes such as methylene norbornene, ethylidene norbornene and isopropylidene norbornene. Specific examples of the preferred compounds (i) and (ii) include 2-methyl-3-methyl-2-[(3-methylbicyclo[2.2.1]hepto-2-yl]bicyclo[2.2.1]heptane.

Specific examples of the alkane derivatives having 2 or more cyclohexane rings (iii) include 2,4-dicyclohexyl-2-methyl pentane and 2,4-dicyclohexyl pentane.

Specific examples of the alkane derivatives having one or more decalin rings and one or more cyclohexane rings (iv) include 1-cyclohexyl-1-decalyl ethane.

The above compounds all exhibit not only a high CED at 40° C. but also, at the same time, a high traction coefficient. For example, the CED at 40° C. and the traction coefficient at 100° C. of some of the above-exemplified compounds are shown in Table 1 below.

Meanwhile, the CED is the value calculated from the above formula (1), and the traction coefficient is the value measured by the experimental method (experiment III) defined in the below-mentioned Examples.

TABLE 1

| | Base | Base | Base | Base | Base | Base |
|---|---|---|---|---|---|---|
| Items | oil 1 | oil 2 | oil 3 | oil 4 | oil 5 | oil 6 |
| Kinematic viscosity at 40° C. | 21.8 | 19.9 | 12.2 | 18.4 | 34.3 | 28.2 |
| (mm²/g) | | | | | | |
| Density at 40° C. (g/cm³) | 0.946 | 0.885 | 0.876 | 0.907 | 0.929 | 0.894 |
| Molecular weight (g/mol) | 246 | 250 | 236 | 277 | 248 | 287 |
| CED at 40° C. (GPa) | 0.234 | 0.213 | 0.211 | 0.198 | 0.238 | 0.197 |
| Traction coefficient (100° C.) | 0.1022 | 0.0973 | 0.0738 | 0.0679 | 0.098 | 0.0653 |

Note:
Base oil 1: 2-methyl-3-methyl-2-[(3-methylbicyclo[2.2.1]hepto-2-yl)methyl]bicyclo[2.2.1]heptane
Base oil 2: 2,4-dicyclohexyl-2-methyl pentane
Base oil 3: 2,4-dicyclohexyl pentane
Base oil 4: Mixture of 75% by mass of the base oil 1 and 25% by mass of poly-α-olefin
Base oil 5: 1-cyclohexyl-1-decalyl ethane
Base oil 6: Mixture of 75% by mass of the base oil 5 and 25% by mass of poly-α-olefin

As is apparent from Table 1, the CED and traction coefficient of the respective base oils have an correlation with each other, i.e., the higher the CED, the higher the traction coefficient becomes. When the CED at 40° C. of the base oil is 0.180 GPa or more, the base oil itself exhibits a traction coefficient at 100° C. of 0.06 or more.

The base oil as the component (A) may be composed of the above synthetic alicyclic (cyclic saturated) hydrocarbon compound solely, or may be in the form of a mixture of the hydrocarbon compound with a mineral oil or a chain hydrocarbon compound. Examples of the mineral oil include paraffinic mineral oils and naphthenic mineral oils. Examples of the chain hydrocarbon compound include poly-α-olefins.

The content of the mineral oil or chain hydrocarbon compound in the base oil is not particularly limited as long as the CED at 40° C. of the base oil is 0.180 GPa or more, and is preferably in the range of 50% by volume or less on the basis of the base oil.

In the lubricating oil composition for continuously variable transmission according to the present invention, the base oil as the component (A) is blended with the component (B) and/or at least one component (C) as explained below.

The component (B) used in the present invention is a phosphoric ester containing a hydrocarbon group having a thioether bond. The phosphoric ester is not limited to particular ones. Examples of the preferred phosphoric ester include acid phosphoric esters and phosphorous esters represented by the following general formula (II):

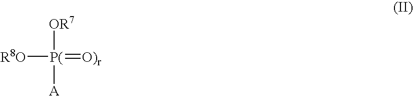

In the general formula (II), r is 0 or 1, and when r is 0, A is a hydroxyl group, whereas when r is 1, A is a hydrogen atom or a hydroxyl group. R⁷ and R⁸ are respectively a hydrogen atom or a hydrocarbon group having 1 to 18 carbon atoms which may have one or more thioether bonds. Examples of the hydrocarbon group having 1 to 18 carbon atoms include a linear or branched alkyl group having 1 to 18 carbon atoms, a cycloalkyl group having 3 to 18 carbon atoms, a linear or branched alkenyl group having 2 to 18 carbon atoms, an aryl group having 6 to 18 carbon atoms and an aralkyl group having 7 to 18 carbon atoms. Specific examples of the alkyl group having 1 to 18 carbon atoms include methyl, ethyl, n-propyl, isopropyl, n-butyl, isobutyl, sec-butyl, tert-butyl, pentyl, hexyl, octyl, 2-ethylhexyl, decyl, dodecyl, tetradecyl, hexadecyl and octadecyl. Examples of the cycloalkyl group having 3 to 18 carbon atoms include cyclopentyl, cyclohexyl, methyl cyclohexyl and cyclooctyl. Examples of the alkenyl group having 2 to 18 carbon atoms include allyl, propenyl, butenyl, octenyl, decenyl and oleyl. Examples of the aryl group having 6 to 18 carbon atoms include phenyl, tolyl, xylyl and naphthyl. Examples of the aralkyl group having 7 to 18 carbon atoms include benzyl, phenethyl and naphthylmethyl.

Examples of the hydrocarbon group having 1 to 18 carbon atoms which contains one or more thioether bonds include hexylthiomethyl, hexylthioethyl, octylthiomethyl, octylthioethyl, dodecylthiomethyl, dodecylthioethyl, hexadecylthiomethyl and hexadecylthioethyl. The R⁷ and R⁸ groups may be the same or different from each other, but at least one of the R⁷ and R⁸ groups is the hydrocarbon group having one or more thioether bonds.

Among the compounds represented by the general formula (II), examples of the acid phosphoric ester include those compounds having a structure represented by the general formula (II-a): 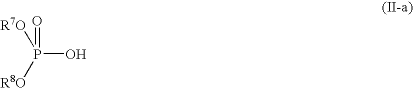
wherein R⁷ and R⁸ are the same as defined above. Examples of the acid phosphoric ester represented by the general formula (II-a) include mono- or di-(hexylthioethyl)hydrogen phosphate, mono- or di-(octylthioethyl)hydrogen phosphate, mono- or di-(dodecylthioethyl)hydrogen phosphate and mono- or di-(hexadecylthioethyl)hydrogen phosphate.

Also, among the compounds represented by the general formula (II), examples of the phosphorous ester include phosphorous esters having a structure represented by the general formula (II-b) or (II-c): 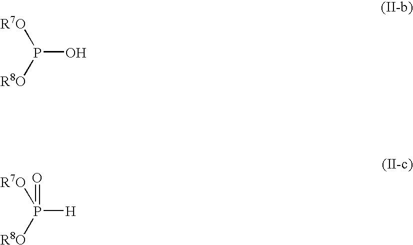
wherein R⁷ and R⁸ are the same as defined above. Examples of the acid phosphorous ester represented by the general formula (II-b) or (II-c) include mono- or di-(hexylthioethyl)hydrogen phosphite, mono- or di-(octylthioethyl)hydrogen phosphite, mono- or di-(dodecylthioethyl)hydrogen phosphite and mono- or di-(hexadecylthioethyl)hydrogen phosphite.

In the present invention, the phosphoric esters containing a hydrocarbon group having a thioether bond as the component (B) may be used alone or in combination of any two or more thereof.

The component (B) serves for enhancing a wear resistance upon contact between metals and at the same time increasing a metal-to-metal friction coefficient, in the presence of the base oil as the component (A).

The amount of the component (B) blended in the composition of the present invention is 50 to 600 ppm by mass and preferably 100 to 400 ppm by mass in terms of the phosphorus content on the basis of the composition. When the amount of the component (B) blended lies within the range of 50 to 600 ppm by mass in terms of the phosphorus content, the resultant composition can ensure a good wear resistance and can be prevented from undergoing deterioration in oxidation stability.

The component (C) used in the present invention is at least one compound selected from the group consisting of phosphoric esters and amine salts thereof, and an overbased calcium sulfonate. These compounds as the component (C) may be used alone or in the form of a mixture of any two or more thereof.

The phosphoric esters used as the component (C) are not particularly limited. Examples of the phosphoric esters as the component (C) include orthophosphoric esters, phosphorous esters, acid phosphoric esters, acid phosphorous esters, and amine salts thereof except for the phosphoric esters as the component (B).

Specific examples of the orthophosphoric esters and phosphorous esters include phosphoric esters and phosphorous esters containing an alkyl or alkenyl group having 1 to 30 carbon atoms such as tributyl phosphate and phosphite, trihexyl phosphate and phosphite, tri-2-ethylhexyl phosphate and phosphite, tridecyl phosphate and phosphite, trilauryl phosphate and phosphite, trimyristyl phosphate and phosphite, tripalmityl phosphate and phosphite, tristearyl phosphate and phosphite and trioleyl phosphate and phosphite; and phosphoric esters and phosphorous esters containing an aryl group having 6 to 30 carbon atoms such as triphenyl phosphate and phosphite, and tricresyl phosphate and phosphite. Specific examples of the acid phosphoric esters and acid phosphorous esters include acid phosphoric esters and acid phosphorous esters containing an alkyl or alkenyl group having 1 to 30 carbon atoms such as mono- or di-butyl hydrogen phosphate and phosphite, mono- or di-pentyl hydrogen phosphate and phosphite, mono- or di-2-ethylhexyl hydrogen phosphate and phosphite, mono- or di-palmityl hydrogen phosphate and phosphite, mono- or di-lauryl hydrogen phosphate and phosphite, mono- or di-stearyl hydrogen phosphate and phosphite and mono- or di-oleyl hydrogen phosphate and phosphite; and acid phosphoric esters and acid phosphorous esters containing an aryl group having 6 to 30 carbon atoms such as mono- or di-phenyl hydrogen phosphate and phosphite and mono- or di-cresyl hydrogen phosphate and phosphite.

The amine salts of the phosphoric esters used as the component (C) in the present invention are amine salts of the above-exemplified phosphoric esters. Examples of amines forming the amine salts include mono-substituted amines, di-substituted amines or tri-substituted amines represented by the general formula (III):
R⁹ₛNH₃₋ₛ (III)
wherein R⁹ is an alkyl or alkenyl group having 3 to 30 carbon atoms, an aryl or aralkyl group having 6 to 30 carbon atoms, or a hydroxyalkyl group having 2 to 30 carbon atoms; s is 1, 2 or 3; and when s is 2 or more, a plurality of the R⁹ groups may be the same or different from each other. In the above general formula (III), the alkyl or alkenyl group having 3 to 30 carbon atoms as R⁹ may be either linear, branched or cyclic.

Examples of the mono-substituted amines include butyl amine, pentyl amine, hexyl amine, cyclohexyl amine, octyl amine, lauryl amine, stearyl amine, oleyl amine and benzyl amine. Examples of the di-substituted amines include dibutyl amine, dipentyl amine, dihexyl amine, dicyclohexyl amine, dioctyl amine, dilauryl amine, distearyl amine, dioleyl amine, dibenzyl amine, stearyl monoethanol amine, decyl monoethanol amine, hexyl monopropanol amine, benzyl monoethanol amine, phenyl monoethanol amine and tolyl monopropanol amine. Examples of the tri-substituted amines include tributyl amine, tripentyl amine, trihexyl amine, tricyclohexyl amine, trioctyl amine, trilauryl amine, tristearyl amine, trioleyl amine, tribenzyl amine, dioleyl monoethanol amine, dilauryl monopropanol amine, dioctyl monoethanol amine, dihexyl monopropanol amine, dibutyl monopropanol amine, oleyl diethanol amine, stearyl dipropanol amine, lauryl diethanol amine, octyl dipropanol amine, butyl diethanol amine, benzyl diethanol amine, phenyl diethanol amine, tolyl dipropanol amine, xylyl diethanol amine, triethanol amine and tripropanol amine.

Among these phosphoric esters and amine salts thereof, in view of enhancing the wear resistance upon contact between metals and, at the same time, increasing the metal-to-metal friction coefficient as aimed by the present invention, preferred are those phosphoric esters containing an alkyl group having 3 to 12 carbon atoms or an aryl group having 6 to 12 carbon atoms, and amine salts thereof, and more preferred are tricresyl phosphate, mono- or di-2-ethylhexyl hydrogen phosphate, mono- or di-2-ethylhexyl hydrogen phosphite and dimethyl acid phosphate lauryl amine.

In the present invention, these phosphoric esters and amine salts thereof may be used alone or in combination of any two or more thereof. The amount of the phosphoric esters and amine salts thereof blended is preferably 50 to 1500 ppm by mass and more preferably 80 to 1000 ppm by mass in terms of the phosphorus content on the basis of the composition. When the amount of the phosphoric esters and amine salts thereof blended is 50 to 1500 ppm by mass in terms of the phosphorus content, the resultant composition can ensure a good wear resistance, etc.

Also, the overbased calcium sulfonate which is one of the compounds used as the component (C) in the present invention, preferably has a base value (hydroxyl value) of 50 to 700 mg KOH/g and more preferably 200 to 600 mg KOH/g. When the base value of the overbased calcium sulfonate is within the range of 50 to 700 mg KOH/g, the resultant composition can exhibit a sufficient effect of enhancing the wear resistance upon contact between metals. In the present invention, the amount of the overbased calcium sulfonate blended is preferably 100 to 2000 ppm by mass and more preferably 200 to 1500 ppm by mass in terms of the calcium content on the basis of the composition. When the amount of the overbased calcium sulfonate blended lies within 100 to 2000 ppm by mass in terms of the calcium content, the resultant composition can exhibit a sufficient effect of enhancing the wear resistance upon contact between metals.

The composition of the present invention may further contain an anti-wear agent as a component (D). The lubricating oil composition for CVT further containing the component (D) can be further improved in durability. Examples of the anti-wear agent include sulfides of animal and vegetable oils and synthetic oils such as sulfurized oils and fats, sulfurized olefins, polysulfides, sulfurized mineral oils, thiophosphoric acids, thiocarbamic acids, thioterpenes and dialkyl thiodipropionates. Specific examples of the sulfurized oils and fats include sulfurized lard, sulfurized rapeseed oils, sulfurized castor oils, sulfurized soybean oils, sulfurized rice bran oils, fatty acid disulfies such as oleic sulfide, and sulfurized esters such as sulfurized methyl oleate. Examples of the sulfurized olefins include those compounds obtained by reacting olefins having 3 to 20 carbon atoms or dimers, trimers and tetramers thereof, with a sulfurizing agent such as sulfur, sulfur chloride and other sulfur halides. Examples of the preferred olefins forming the sulfurized olefins include propylene, isobutene and diisobutene. The polysulfides are dihydrocarbyl polysulfides represented by the general formula (IV):
R¹⁰—Sₓ—R¹¹ (IV)
wherein R¹⁰ and R¹¹ are respectively an alkyl group having 1 to 20 carbon atoms, an aryl group having 6 to 20 carbon atoms, an alkylaryl group having 7 to 20 carbon atoms or an arylalkyl group having 7 to 20 carbon atoms, and may be the same or different from each other; x is a real number (more specifically, rational number) of 2 to 8.

Specific examples of R¹⁰ and R¹¹ in the general formula (IV) include methyl, ethyl, n-propyl, isopropyl, n-butyl, isobutyl, sec-butyl, t-butyl, various pentyl groups, various hexyl groups, various heptyl groups, various octyl groups, various nonyl groups, various decyl groups, various dodecyl groups, cyclohexyl, cyclooctyl, phenyl, naphthyl, tolyl, xylyl, benzyl and phenethyl. Examples of the preferred polysulfides include dibenzyl polysulfide, di-t-nonyl polysulfide and didodecyl polysulfide. Examples of the dithiophosphoric acids include zinc dialkyldithiophosphates and molybdenum dialkyldithiophosphates. Examples of the thiocarbamic acids include zinc dialkyldithiocarbamates and molybdenum dialkyldithiocarbamates. Examples of the thioterpenes include reaction products of phosphorus pentasulfide and pinene. Examples of the dialkylthiodipropionates include dilaurylthiodipropionate and distearylthiodipropionate. Among these sulfur containing anti-wear agents, preferred are sulfurized lard, zinc dialkyldithiophosphates, dilaurylthiodipropionates and isobutene sulfide, and more preferred are zinc dialkyldithiophosphates.

In the present invention, these sulfur containing anti-wear agents may be used alone or in combination of any two or more thereof. The content of the thiophosphates or the thiocarbamates as the sulfur containing anti-wear agent is preferably 0.2 to 2.0% by mass and more preferably 0.5 to 1.0% by mass on the basis of the composition. The content of the sulfur containing anti-wear agent other than the thiophosphates or the thiocarbamates is preferably 100 to 5000 ppm by mass and more preferably 400 to 3000 ppm by mass in terms of the sulfur content on the basis of the composition. When the content of the sulfur containing anti-wear agent lies within the above-specified range, the resultant composition can exhibit a good effect of improving an anti-seizing property and a wear resistance upon contact between metals.

The lubricating oil composition for CVT according to the present invention can be obtained by blending the base oil as the component (A) with the component (B) and/or the component (C) mentioned above. In the more preferred embodiment of the present invention, the lubricating oil composition for CVT is obtained by blending the component (A) with both the component (B) and the component (C). The lubricating oil composition according to the more preferred embodiment of the present invention can exhibit a still higher total friction coefficient as well as a high wear resistance, so that a power transmission capacity of the continuously variable transmission can be more increased and the effect of keeping the high power transmission capacity can be attained.

The effect and mechanism of the present invention which can provide the above advantages are considered as follows. The friction coefficient observed on a power-transmitting surface of CVT is represented by the following formula (2):
μₜₒₜₐₗ=m·μₘ+(1−m)·μₜ (2)
wherein μₜₒₜₐₗ is a total friction coefficient; μₘ is a metal-to-metal friction coefficient; μₜ is a traction coefficient; and m is a ratio (Nₘ/N) of a load (Nₘ) supported by a contact portion between metals to whole load (N=Nₘ+Nₜ; and (1−m) is a ratio (Nₜ/N) of a load (Nₜ) supported by an oil film to the whole load.

Accordingly, if the load (pressing force) applied to the power-transmitting surface of CVT is kept constant and a sharing ratio of the load is kept constant, the total friction coefficient μₜₒₜₐₗ is influenced by the metal-to-metal friction coefficient μₘ at the surface and the traction coefficient μₜ of the EHL oil film.

The present inventors have found that the base oil having a high cohesive energy density (CED) exhibits a high traction coefficient, and has such an effect of enhancing the metal-to-metal friction coefficient μm and the wear resistance which are imparted by the components (B) and/or (C).

Therefore, by combining the base oil as the component (A) having a high traction coefficient μₜ with the components (B) and/or (C) having a high metal-to-metal friction coefficient μₘ enhanced by the component (A), the total friction coefficient μₜₒₜₐₗ can also be enhanced.

Further, when using the components (B) and/or (C) having the enhanced wear resistance, the power-transmitting surface can be inhibited from undergoing wear, so that the configuration of the power-transmitting surface such as surface roughness can be kept in an initial condition thereof. As a result, since the metal-to-metal friction coefficient, the traction coefficient and other factors remain unchanged, the total friction coefficient can be prevented from being deteriorated. For these reasons, it is suggested that the power transmission capacity is enhanced, and is inhibited from being lowered.

The lubricating oil composition for CVT according to the present invention may further contain known additives unless the addition thereof adversely affects the objects of the present invention. Examples of the additives include detergent dispersants such as succinimide and boron-based succinimide; antioxidants such as phenol-based compounds and amine-based compounds; corrosion inhibitors such as benzotriazole-based compounds and thiazole-based compounds; rust preventives such as metal sulfonate-based compounds and succinic ester-based compounds; defoaming agents such as silicone-based compounds and fluorinated silicone-based compounds; and viscosity index improver such as polymethacrylate-based compounds and olefin copolymers. The content of the additives blended may be appropriately determined according to the objects and applications of the resultant composition. The total amount of the additives blended is usually 20% by mass or less on the basis of the composition.

### EXAMPLES

The present invention is described in more detail by referring to the following examples and comparative examples. However, it should be noted that these examples are only illustrative and not intended to limit the invention thereto. Meanwhile, various properties of the CVT lubricating oil were measured by the following methods.

(1) Experiment I: Measurement of Friction Coefficient

A block-on-ring type friction test (LFW1 test) was conducted under the following friction conditions to measure a total friction coefficient after 5 min from initiation of the test at respective slide velocities.

Load: 1110 N (average Hertz stress upon initiation of the test: 0.49 GPa)

Oil temperature: 110° C.

Test piece: Ring (φ35×5; SAE01 steel; RC60), Block (SAE4620 steel; RC60)

Slide velocity: 0.13 m/s, 0.25 m/s, 0.5 m/s and 1 m/s

(2) Experiment II: Measurement of Friction Coefficient and Wear Resistance

A block-on-ring type friction test was conducted under the following friction conditions to measure total friction coefficients immediately after initiation of the test and after 60 min therefrom, and a width of wear on the block after the elapse of 60 min.

Load: 1530 N (average Hertz stress upon initiation of the test: 0.57 GPa)

Oil temperature: 130° C.

Test piece: Ring (φ35×5; SAE01 steel; RC60), Block (SAE4620 steel; RC60)

Slide velocity: 0.37 m/s

(3) Experiment III: Measurement of Traction Properties

The traction properties were measured by a twin-cylinder tester. A pair of metallic cylinders (material: bearing steel/SUJ-2; φ40/flat×φ40/r20; hardness: RC61; surface roughness Rm: 0.03) were disposed in an opposed relation to each other, and both rotated at 3300 rpm while applying a load of 147.1 N (average Hertz stress: 0.83 GPa) such that the difference in slide/roll ratio S.R. (=(U1−U2)×100/(U1+U2) wherein U1 and U2 are velocities of the respective cylinders between both the cylinders) is 5%, to measure a tangential force F (traction force N) generated at a contact portion between the two cylinders, thereby determining a traction coefficient μₜ (=F/147.1).

### Example 1 and Comparative Examples 1 to 3

Using base oils and additives shown in Table 2, the lubricating oil composition for CVT according to the present invention (Example 1) and the comparative lubricating oil compositions for CVT (Comparative Examples 1 to 3) as shown in Table 3 were prepared to measure total friction coefficients thereof by the method described in the above Experiment I as well as traction coefficients thereof by the method described in the above Experiment III. The results of the measurement are shown in Table 3.

TABLE 2-1

| | | CED at 40° C. |
|---|---|---|
| Base oil | Composition | (GPa) |
| Base oil 1 | 2-methyl-3-methyl-2-[(3-methylbicyclo[2.2.1] | 0.234 |
| | hepto-2-yl)methyl]bicyclo[2.2.1]heptane | |
| Base oil 2 | Naphthenic mineral oil (kinematic viscosity at | 0.178 |
| | 40° C.: 27.9 mm²/s) | |
| Base oil 3 | Paraffinic mineral oil (kinematic viscosity at 40° C | 0.128 |
| | 20.3 mm²/s) | |
| Base oil 4 | Poly-α-olefin (kinematic viscosity at 40° C.: 30.0 | 0.101 |
| | mm²/s) | |

TABLE 2-2

| Additive | Composition |
|---|---|
| Additive 1 | Di(octylthioethyl)hydrogen phosphite |
| Additive 2 | Boron-containing imide-based dispersant “ECA5025” available |
| | from Infineum Inc. (nitrogen content: 1.35% by mass; boron |
| | content: 0.35% by mass) |
| Additive 3 | Overbased calcium sulfonate “Bryton C400” available from |
| | Witoco Chemical Inc. (base value: 400 mg KOH/g; Ca content: |
| | 15.2% by mass; S content: 1.2% by mass) |
| Additive 4 | Reaction product of α-pinene and phosphorus pentasulfide “Hitec |
| | 649” available from Ethyl Japan Co., Ltd. (total acid value: 6.0 |
| | mg KOH/g; S content: 12.5% by mass; P content: 4.8% by mass) |

TABLE 3

| Example | Comparative Examples | | |
|---|---|---|---|
| 1 | 1 | 2 | 3 |

Blending ratio (wt %)

| | | | | |
|---|---|---|---|---|
| Base oil 1 | 97.1 | | | |
| Base oil 2 | | 97.1 | | |
| Base oil 3 | | | 97.1 | |
| Base oil 4 | | | | 97.1 |
| Additive 1 | 0.4 | 0.4 | 0.4 | 0.4 |
| Additive 2 | 2.5 | 2.5 | 2.5 | 2.5 |
| Experimental results | | | | |
| Traction coefficient at | 0.0998 | 0.0552 | 0.0120 | 0.0050 |
| 100° C. | | | | |
| Total friction | | | | |
| coefficient | | | | |
| Slide velocity: 0.13 | 0.118 | 0.113 | 0.099 | 0.107 |
| m/s | | | | |
| Slide velocity: 0.25 | 0.113 | 0.109 | 0.093 | 0.1 |
| m/s | | | | |
| Slide velocity: 0.5 m/s | 0.106 | 0.102 | 0.086 | 0.092 |
| Slide velocity: 1.0 m/s | 0.102 | 0.096 | 0.076 | 0.082 |

### Examples 2 to 4 and Comparative Examples 4 to 13

Using base oils and additives shown in Table 2, the lubricating oil compositions for CVT according to the present invention (Examples 2 to 4) and the comparative lubricating oil compositions for CVT (Comparative Examples 4 to 13) as shown in Table 4 were prepared to measure total friction coefficients, wear widths and traction coefficients thereof by the methods described in the above Experiments I, II and III. The results of the measurement are shown in Table 4.

TABLE 4-1

| | Examples | | Comparative Examples | | |
|---|---|---|---|---|---|
| | 2 | 3 | 4 | 5 | 6 |
| Blending ratio (wt %) | | | | | |
| Base oil 1 | 99.6 | 97.1 | | | |
| Base oil 2 | | | 97.1 | | |
| Base oil 3 | | | | 97.1 | |
| Base oil 4 | | | | | 97.1 |
| Additive 1 | 0.4 | 0.4 | 0.4 | 0.4 | 0.4 |
| Additive 2 | | 2.5 | 2.5 | 2.5 | 2.5 |
| Additive 3 | | | | | |
| Additive 4 | | | | | |
| Experimental results | | | | | |
| Traction coefficient at | 0.1020 | 0.0998 | 0.0552 | 0.0120 | 0.0050 |
| 100° C. | | | | | |
| Total friction | | | | | |
| coefficient | | | | | |
| Immediately after | 0.13 | 0.133 | 0.123 | 0.117 | 0.117 |
| initiation of the test | | | | | |
| After 60 min from | 0.114 | 0.118 | 0.112 | 0.108 | 0.107 |
| initiation of the test | | | | | |
| Wear width in block | 0.71 | 0.75 | 0.74 | 0.83 | 0.82 |
| (mm) | | | | | |

TABLE 4-2

| | Comparative Examples | | |
|---|---|---|---|
| Example 4 | 7 | 8 | 9 |

Blending ratio (wt %)

| | | | | |
|---|---|---|---|---|
| Base oil 1 | 96.9 | | | |
| Base oil 2 | | 96.9 | | |
| Base oil 3 | | | 96.9 | |
| Base oil 4 | | | | 96.9 |
| Additive 1 | 0.4 | 0.4 | 0.4 | 0.4 |
| Additive 2 | 2.5 | 2.5 | 2.5 | 2.5 |
| Additive 3 | 0.2 | 0.2 | 0.2 | 0.2 |
| Additive 4 | | | | |
| Experimental results | | | | |
| Traction coefficient at | 0.0996 | 0.0550 | 0.0118 | 0.0050 |
| 100° C. | | | | |
| Total friction | | | | |
| coefficient | | | | |
| Immediately after | 0.145 | 0.138 | 0.125 | 0.122 |
| initiation of the test | | | | |
| After 60 min from | 0.128 | 0.12 | 0.116 | 0.114 |
| initiation of the test | | | | |
| Wear width in block | 0.81 | 0.78 | 0.83 | 0.85 |
| (mm) | | | | |

TABLE 4-3

| Comparative Examples | | | |
|---|---|---|---|
| 10 | 11 | 12 | 13 |

Blending ratio (wt %)

| | | | | |
|---|---|---|---|---|
| Base oil SN3 | 99.5 | | | |
| Base oil MN1 | | 99.5 | | |
| Base oil M02 | | | 99.5 | |
| Base oil SP3 | | | | 99.5 |
| Additive 1 | | | | |
| Additive 2 | | | | |
| Additive 3 | | | | |
| Additive 4 | 0.5 | 0.5 | 0.5 | 0.5 |
| Experimental results | | | | |
| Traction coefficient at | 0.1020 | 0.0555 | 0.0121 | 0.0050 |
| 100° C. | | | | |
| Total friction | | | | |
| coefficient | | | | |
| Immediately after | 0.135 | 0.123 | 0.122 | 0.12 |
| initiation of the test | | | | |
| After 60 mim from | 0.098 | 0.093 | 0.09 | 0.084 |
| initiation of the test | | | | |
| Wear width in block | 1.15 | 1.04 | 1.11 | 1.13 |
| (mm) | | | | |

### INDUSTRIAL APPLICABILITY

The lubricating oil composition for CVT according to the present invention can be used as a general-purpose lubricating oil composition for metallic belt type CVT, chain type CVT and traction drive type CVT in order to increase a power transmission capacity thereof and enhance a durability thereof.

1. A lubricating oil composition for continuously variable transmission comprising:
(A) a base oil comprising a hydrocarbon compound having a cohesive energy density at 40° C. of 0.180 GPa or more, said base oil having a kinematic viscosity at 40° C. of 5 to 150 mm²/s, and
(B) a phosphoric ester containing a hydrocarbon group having a thioether bond, and/or (C) at least one compound selected from the group consisting of phosphoric esters and amine salts thereof, and an overbased calcium sulfonate.

2. A lubricating oil composition for continuously variable transmission according to claim 1, wherein said cohesive energy density (CED) at 40° C. is calculated from the following formula (1):
CED(GPa)=0.0204(d/MW)·T·ln(2.51ηMW) (1)
wherein d is a density (g/cm³) at 40° C.; MW is a molecular weight (g/mol); T is an absolute temperature (K); and η is a kinematic viscosity (mm²/s) at 40° C.

3. A lubricating oil composition for continuously variable transmission according to claim 1 or 2, wherein said base oil (A) contains at least one compound selected from the group consisting of (i) dimerized norbornanes, (ii) hydrogenated dimers, trimers and tetramers of norbornanes and/or norbornenes, (iii) alkane derivatives having 2 or more cyclohexane rings, and (iv) alkane derivatives having one or more decalin rings and one or more cyclohexane rings.

4. A lubricating oil composition for continuously variable transmission according to any one of claims 1 to 3, wherein said phosphoric ester (B) containing a hydrocarbon group having a thioether bond is an acid phosphoric ester or phosphorous ester represented by the general formula (II): 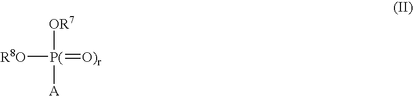
wherein r is 0 or 1; when r is 0, A is a hydroxyl group, and when r is 1, A is a hydrogen atom or a hydroxyl group; and R⁷ and R⁸ are respectively a hydrogen atom or a hydrocarbon group having 1 to 18 carbon atoms which may have one or more thioether bonds, and at least one of R⁷ and R⁸ is the hydrocarbon group having a thioether bond.

5. A lubricating oil composition for continuously variable transmission according to any one of claims 1 to 4, wherein said phosphoric esters and amine salts thereof as the component (C) are phosphoric esters containing an alkyl group having 3 to 12 carbon atoms or an aryl group having 6 to 12 carbon atoms, and amine salts thereof.

6. A lubricating oil composition for continuously variable transmission according to any one of claims 1 to 5, wherein said overbased calcium sulfonate as the component (C) has a base value of 50 to 700 mg KOH/g.

7. A lubricating oil composition for continuously variable transmission according to any one of claims 1 to 6, further comprising (D) a sulfur containing anti-wear agent.

8. A lubricating oil composition for continuously variable transmission according to any one of claims 1 to 7, wherein said continuously variable transmission is of a metallic belt type.

9. A lubricating oil composition for continuously variable transmission according to any one of claims 1 to 7, wherein said continuously variable transmission is of a chain type.

10. A lubricating oil composition for continuously variable transmission according to any one of claims 1 to 7, wherein said continuously variable transmission is of a traction drive type.
